(19) United States
(12) Patent Application Publication
(10) Pub. No.: US 2014/0024711 A1
(43) Pub. Date: Jan. 23, 2014

(54) LOW ALCOHOL ANTIMICROBIAL CLEANSING COMPOSITION

(71) Applicants: Torsten Hedbom, Falun (SE); Stefan Snell, Falun (SE)

(72) Inventors: Torsten Hedbom, Falun (SE); Stefan Snell, Falun (SE)

(73) Assignee: SCA HYGIENE PRODUCTS AB, Goteborg (SE)

(21) Appl. No.: 14/003,068

(22) PCT Filed: Mar. 8, 2011
(86) PCT No.: PCT/EP2011/053448

§ 371 Date: Oct. 3, 2013

(51) Int. Cl. A61K 8/04; A01N 25/16; A61Q 19/10; A47K 5/14

(52) U.S. Cl. CPC A61K 8/046; A47K 5/14; A01N 25/16; A61Q 19/10
USPC 514/556; 222/190; 514/724

(57) ABSTRACT

An antimicrobial cleansing composition including (a) 20 to less than 60 wt. % of at least one alcohol selected from ethanol and propanol; (b) 0.5 to 10 wt. % of a surfactant selected from sulfate salt surfactants and amphoteric surfactants. The composition shows effective cleansing and antimicrobial properties. Hence, using this composition, it will no longer be required to provide in hospitals two separate dispensers one for foam soap and one for antimicrobial alcohol-based composition. The antimicrobial cleansing composition can be used for making foam. Also, a dispenser contains the above antimicrobial cleansing composition.

### CROSS-REFERENCE TO PRIOR APPLICATION

This application is a §371 National Stage Application of PCT International Application No. PCT/EP2011/053448 filed Mar. 8, 2011, which is incorporated herein in its entirety.

The present disclosure relates to an antimicrobial cleansing composition which can be used to generate cleansing foam and which has a high antimicrobial efficacy despite the relatively low alcohol content.

### TECHNICAL BACKGROUND

In hospitals and doctors' practices, it is of utmost importance to work in a germ-free environment when patients are treated. For this reason, physicians, nurses and other medical staff are obligated to frequently wash and disinfect their hands. Customarily, this is done by first washing and cleansing the hands using a detergent-based liquid soap followed by a second wash involving the wetting and rubbing of the hands with a disinfectant composition. In hospitals and doctors' practices, there are typically two dispensers: one for the liquid soap and one for the antibacterial composition, which are mounted to the wall above the wash basins.

As a rule, commercially available disinfectant compositions are alcohol-based and contain ethanol and/or isopropyl alcohol in amounts of more than 60% by weight. It is generally believed in the art that these compositions require a fairly high alcohol content to be antibacterial. However, since long term use of high ethanol disinfectant compositions can be associated with skin irritation, there is a need for disinfectant compositions having a lower alcohol content. Furthermore, it would be desirable to reduce the time and effort required for cleansing and disinfecting the hands in hospitals and doctors' practices.

US 2002/0077257 A1 discloses a sanitizing hand cleanser including approximately 60 wt. % to approximately 90 wt. % of one or more organic alcohols, approximately 5 wt. % to approximately 35 wt. % of one or more silicone-based materials, one or more thickening agents and optional humectants and skin pH-maintaining additives.

### SUMMARY

It is desired to provide an alcohol-based composition which has good antimicrobial and cleansing properties.

It is further desired to provide an antimicrobial cleansing composition which does not irritate the skin.

According to one embodiment, it is desired to provide an antimicrobial cleansing composition able to be dispensed as a sufficiently stable foam.

Further benefits will become apparent from the following description of the embodiments.

Whenever the present description and the claims define a composition as “comprising” certain ingredients, the respective composition may also “consist of” these ingredients in a further embodiment. If the terms “optionally” or “may” are used, this indicates that the corresponding ingredient can also be absent from the respective composition. Hereinafter, the unit “wt. %” refers to the total weight of the antimicrobial cleansing composition.

The present disclosure provides an antimicrobial cleansing composition comprising
(a) 20 to less than 60 wt. %, 25 to 55 wt. %, or 35 to 50 wt. % of at least one alcohol selected from ethanol and propanol,
(b) 0.5 to 10 wt. % of a surfactant selected from sulfate salt surfactants and amphoteric surfactants, such as a sulfate salt surfactant comprising (i) an anionic molecule comprising a monovalent hydrocarbon group having 8 to 20 carbon atoms, which is linked via an alkyleneoxy unit or polyalkyleneoxy unit with an anionic group and (ii) as cationic counterion an alkyl group having 2 to 4 carbon atoms and comprising at least one amino group and at least one hydroxyl group substituent, and
(c) water.

It is a characteristic feature of the compositions that they provide antimicrobial and cleansing action at the same time.

In this case, the term “antimicrobial” means the capacity to kill or inhibit the growth of microorganisms selected from bacteria, viruses and fungi.

This antimicrobial cleansing composition can be provided as a transparent or translucent aqueous solution. Hence, it normally comprises one single aqueous-alcoholic phase but no further phases, for instance of oil type.

In a further embodiment, this antimicrobial cleansing composition comprises
(a) 20 to less than 60 wt. %, 25 to 55 wt. %, or 35 to 50 wt. % of an alcohol selected from ethanol and propanol,
(b) 0.5 to 10 wt. % of the anionic surfactant described above,
(b′) up to 5 wt. %, or up to 1 wt. % of a secondary surfactant (different from surfactant (b)) said secondary surfactant can be a nonionic co-surfactant,
(c) optionally up to 5 wt. %, e.g. 0.001 to 4 wt. %, of an agent selected from antibacterial, antifungal and antiviral agents,
(d) optionally 0.001 to 5 wt. %, e.g. 0.1 to 3 wt. %, of a thickener,
(e) optionally 0.001 to 8 wt. %, e.g. 0.5 to 5 wt. %, of a humectant,
(f) optionally 0.001 to 8 wt. %, e.g. 0.5 to 5 wt. %, of a film-forming agent,
(g) optionally 0.001 to 8 wt. %, e.g. 0.1 to 5 wt. %, of at least one skin care active,
(h) optionally 0.001 to 3 wt. %, e.g. 0.1 to 1 wt. %, of a hydrophilic solvent,
(i) optionally 0.001 to 3 wt. %, e.g. up to 1 wt. %, of a pH-regulating compound,
(j) optionally 0.001 to 3 wt. %, e.g. up to 1 wt. %, of other auxiliary substances, and
(k) water.

It should be understood that embodiments given for one of components (a), (b), (b′), (c), (d), (e), (f), (g), (h), (i) and (j), respectively, can be used singly or in combination with other embodiments given for the same component. When one of the embodiments given for these components is used singly (as the only embodiment of component (a), (b), etc. present in the composition), the numerical wt. % ranges disclosed in the present application apply to the singly used component as well.

According to one embodiment, this antimicrobial cleansing composition is capable of forming a foam and comprises at least (a) 35 to 50 wt. % of at least one alcohol selected from ethanol and propanol, in particular embodiments, ethanol containing not more than 20 wt. % propanol, and (b) 1 to 8 wt. % of the surfactant described above. In a further embodiment, this foamable composition comprises further (e) 0.5 to 5 wt. % of a humectant, (g) 0.1 to 5 wt. % of at least one skin care active, and optionally one or more of optional components (c), (f), (h), (i) and (j) in the above-stated amounts, the remainder being water.

The present disclosure also relates to the use of this antimicrobial cleansing composition for making foam.

Further, the disclosure relates to a dispenser, in particular embodiments, a foam-generating dispenser containing the above antimicrobial cleansing compositions.

### DETAILED DESCRIPTION OF PREFERRED EMBODIMENTS

The antimicrobial cleansing composition comprises
(a) 20 to less than 60 wt. %, 25 to 55 wt. %, or 35 to 50 wt. % of at least one alcohol selected from ethanol and propanol,
(b) 0.5 to 10 wt. % of a surfactant selected from sulfate salt surfactants and amphoteric surfactants, in particular embodiments, a sulfate salt surfactant comprising (i) an anionic molecule comprising a monovalent hydrocarbon group having 8 to 20 carbon atoms, which is linked via an alkyleneoxy unit or polyalkyleneoxy unit with a sulfate group and (ii) as cationic counterion an alkyl group having 2 to 4 carbon atoms and comprising at least one amino group and at least one hydroxyl group substituent, and
(c) water.

The composition of the disclosure shows a unique combination of cleansing and antimicrobial properties. Hence, using this composition it will no longer be required to provide in hospitals two separate dispensers for one (foam) soap and one antimicrobial alcohol-based composition.

Surprisingly, it was discovered that the use of specific surfactants, e.g. MIPA-C12-14 fatty alcohol polyethyleneglycolether sulfate, in fairly low amounts enhances the antimicrobial effect of alcohol-based disinfectant compositions. This makes it possible to use lower amounts of alcohol than present in conventional antimicrobial cleansing compositions. Both low alcohol and low surfactant content contribute to the mildness of the composition. For this reason, it presents no problem if the user does not completely rinse off antimicrobial composition remaining on his hands.

Further, surfactant (b) increases the capacity of the antimicrobial cleansing composition to form foam. In some embodiments, stable foam can be generated with the use of a non-propellant foam-dispensing device from a non-pressurized container.

According to embodiments, 20 to less than 60 wt. %, e.g. 25 to 55 wt. % of ethanol and/or propanol (n-propanol and/or isopropanol) is used as component (a) together with surfactant (b) to provide the cleansing composition with antimicrobial properties. In particular embodiments, 35 to 50 wt. % ethanol and/or propanol (n-propanol and/or isopropanol) is contained in the composition. In further embodiments, these alcohols are contained in amounts of 35 to 45 wt. %, such as 37 to 43 wt. %. In certain embodiments, ethanol is used. In typical embodiments, “ethanol and/or propanol” is ethanol containing not more than 20 wt. % propanol, based on the total amount of component (a).

The antimicrobial cleansing composition also comprises 0.5 to 10 wt. %, or 1 to 8 wt. %, e.g. from 2 to 6 wt. % or 3 to 5 wt. %, of surfactant (b) which is selected from sulfate salt surfactants and amphoteric surfactants.

In particular embodiments, the sulfate salt surfactant has the following formula (I):

R¹—(O-alkylene)ₙ-OSO₃⁻Z⁺ (I)

wherein n is zero or an integer (e.g. 1 to 10 on average), 1 to 8, 1 to 6, or 1 to 4, each on average.

In particular embodiments, “alkylene” is ethylene or propylene.

Z⁺ is any positive counter-ion, for instance an alkali metal ion such as K′ or Na⁺, an ammonium group (NH⁴) or a protonated amine. Z⁺ may also be selected from quaternary ammonium ions, in particular embodiments, from those having not more than 10 carbon atoms. The protonated amine can be a monoalkylamine, dialkylamine or trialkylamine having up to 18, or up to 12 carbon atoms in total. The carbon number per alkyl group can be up to 6, e.g. 2 to 4 carbon atoms. The N-substituting alkyl may carry further substituents such as hydroxyl as in hydroxyalkyl (alkanol) residues having up to 6, e.g. 2 to 4 carbon atoms. The protonated amine can be a protonated mono-, di- or tri-hydroxyalkylamine which is selected e.g. from monoethanolamine, diethanolamine, triethanolamine, monopropanolamine, dipropanolamine and tripropanolamine. The propanol unit can be an isopropanol unit as in MIPA, DIPA and TIPA.

R¹ is a monovalent hydrocarbon residue having 8 to 24 carbon atoms, or 10 to 16 carbon atoms, such as 12 to 14 carbon atoms. If the surfactant comprises a mixture of hydrocarbon residues having different chain lengths as in many commercially available surfactants the carbon ranges refer to the average carbon number. The monovalent hydrocarbon group may be substituted and/or unsaturated and/or branched. In particular embodiments, it is a linear hydrocarbon group (alkyl group) which neither includes unsaturation nor carries any substituents.

In a certain embodiment, the sulfate salt surfactant comprises (i) an anionic molecule comprising a monovalent hydrocarbon group having 8 to 20 carbon atoms, which is linked via an alkyleneoxy unit or polyalkyleneoxy unit with a sulfate group and (ii) as cationic counter-ion a protonated amine as defined above, or a protonated mono-, di- or tri-hydroxylalkylamine. The protonated mono-, di- or tri-hydroxylalkylamine can be one wherein the alkyl group has 2 to 4 carbon atoms and comprises one hydroxyl group substituent.

It should be understood that the amine typically occurs in protonated form due to proton transfer from the protonated form of the sulfate group present in the anionic molecule portion. The cationic counter-ion present in this particular embodiment is propane substituted by one (protonated) amino group and one hydroxyl group. The amino group and the hydroxyl group can be located on adjacent carbon atoms. In a particular embodiment, the counter-ion is (protonated) 2-hydroxy-1-amino propane (isopropanolamine) as in MIPA-laureth sulfate.

The aforementioned (poly) alkyleneoxy unit can be formed by ethyleneoxy and/or propyleneoxy units. In particular embodiments, the (poly)alkyleneoxy unit is a (poly)ethyleneoxy unit having on average up to 10 units, or up to 6 units.

In a certain embodiment, the anionic molecule portion of the surfactant comprises a C10 to C16 alkyl group, e.g. C12 to C14 alkyl group (average C number in mixtures) which is linked via a (O—CH₂—CH₂)ₙ unit, wherein the average value of n is 1 to 6, or 1 to 4, to a sulfate group. One commercially available representative embodiment of this class of surfactants is MIPA-laureth sulfate having the formula CH₃(CH₂)₁₀CH₂(OCH₂CH₂)ₙOSO₃H.H₂NCH₂C(OH)HCH₃, avg. n=1-4.

Surfactant (b) may also be selected from amphoteric surfactants which can be zwitterionic or not. Among the zwitterionic amphoteric surfactants, those including a betaine unit (betaine=—CH₂—N⁺(CH₃)₂—CH₂—COO⁻) are preferred. In particular embodiments, amphoteric betaine surfactants have the following formula (II):

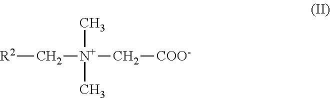

wherein R² is a monovalent substituent having from 8 to 24 carbon atoms (average C number in mixtures) and optionally units selected from —O—, —CO—, —COO—, —NH— and —CO—NH—. In one embodiment, the substituent R² comprises one —CO—NH— unit. R² can be —R⁵—CO—NH—CH₂CH₂— wherein R5 is defined as R1 above. One embodiment of this surfactant type is cocoamidopropyl betaine.

Among the non-zwitterionic amphoteric surfactants those comprising an amphoacetate structure (═N—CH₂—COO⁻Z⁺; Z⁺ as explained above). In one embodiment, these have the following structure (III):

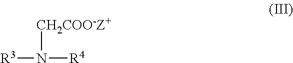

wherein Z⁺ has the same meaning as explained above and R³ and/or R⁴ have the same meaning as indicated above for R². In particular embodiments, R³ and/or R⁴ are R⁵—CO—NH—CH₂CH₂— wherein R⁵ is defined as R¹ above.

If, in one embodiment, only one of R³ and R⁴ has the aforementioned meaning, the other substituent is a hydroxyalkyl residue, such as one with less than 10 carbon atoms such as —CH₂CH₂—OH. One embodiment of this surfactant type is sodium cocoamphoacetate.

The antimicrobial cleansing composition may further comprise one or more components selected from secondary surfactants, antibacterial agents, antiviral agents, antifungal agents, thickening agents, humectants, film-forming agents, skin care actives, hydrophilic solvents, pH-regulating substances and other auxiliary agents typically used in antimicrobial cleansing compositions. In particular embodiments, their total amount does not exceed 25 wt. %.

It is possible to add secondary surfactants (co-surfactants) to all embodiments. Such secondary surfactants (b′), e.g. non-ionic surfactants, may be present in an optional amount of up to 5 wt. %, such as in an amount of up to 1 wt. %, e.g. 0.1 to 1 wt. %. Nonionic co-surfactants such as sugar-based surfactants, e.g. glucoside surfactants, may be added to further increase the efficiency of surfactant (b) and/or enhance the spreading behaviour of the antimicrobial composition and/or better the after-feel of the antimicrobial composition on the skin. Sometime it may also be convenient to use a commercially available surfactant mixture which contains, in addition to surfactant (b), smaller amounts of secondary surfactants (b′).

In view of the excellent antimicrobial properties of the composition, the presence of such secondary surfactants is not required. Hence, in one aspect, surfactant (b) is the sole surfactant.

The antimicrobial cleansing composition may also contain, as component (c), an optional amount of up to 5 wt. % of at least one agent selected from antibacterial, antifungal and antiviral agents. Their addition may be contemplated, for instance, if activity against specific bacterial, fungal or viral strains is to be provided. In view of the excellent antimicrobial activity of the claimed composition, however, it is one aspect not to use such an agent.

The antimicrobial cleansing composition may contain 0.1 to 3 wt. % of a thickener as component (d), particularly in the second embodiment where the ability to form stable foam is not required.

The thickening agents can be selected from agents commonly used in alcohol-based cleansing compositions and cosmetic compositions. In particular embodiments, they belong to the class of hydrocolloids. Suitable thickening agents can be selected from cellulose ethers, polysaccharides and polyacrylates. In one embodiment, the thickener is hydroxypropyl cellulose. However, the use of gelling agents is preferably avoided since commercial alcohol gel products sometimes leave an unpleasant, sticky after-feel.

As a further optional component (e), the antimicrobial cleansing compositions may also contain 0.5 to 5 wt. % of a humectant. The function of humectants is to attract water to the stratum corneum of the human skin. Suitable humectants may be selected from cosmetic ingredients typically used for this purpose. Specifically, the humectant may be selected from polyols such as glycerol, sugar alcohols such as sorbitol, urea, alpha hydroxy acids such as lactic acid, glycolic acid and hyaluronic acid.

As a further optional component (f), film-forming agents may be added in an amount of 0.5 to 5 wt. %. Film-forming agents are capable of inhibiting transepidermal water loss (TWEL) in the stratum corneum. Particular film-forming agents are ethanol-soluble. More particular film-forming agents are ethanol-soluble silicones such as dimethicone.

As a further optional component (g), the antimicrobial cleansing composition may contain 0.1 to 5 wt. % of at least one skin care active. The skin care actives can be obtained from natural sources (plant extracts). They can be selected, for example, from agents having a skin-soothing, antiphlogistic (reduction of skin irritation), wound-healing, and/or cell-regenerating effect. Examples of suitable skin care actives include betaine (trimethylglycine), allantoin; aloe vera extract; chamomile extract containing azulene and bisabolol; echinacea; dragosantanol; panthenol; liquorice root extract containing 18-glycyrrhetinic acid; lime tree extract containing quercetin and/or glyco-rutin; marigold (calendula oil); phytosterols, optionally ethoxylated (available from Henkel under the tradename “Generol”); chitosan (acetylated chitin); anthocyanidins; gingko leaf extract containing quercetin and rutin; horse chestnut containing quercetin and campherol; vitamins or provitamins such as provitamin B5 or Vitamin E; avocado oil; birch extract; arnica; extract of rose of Sharon or St. John's Wort; teatree oil; cucumber, hops or hamamelis extracts or ingredients. The aforementioned extracts can be used singly or in combination. Likewise, the extracts can be substituted for suitable amounts of one or more of their ingredients, as stated above. In certain embodiments, the use of betaine is preferred in view of its cell-regenerating effect.

As a further optional component (h), the antimicrobial cleansing composition may also contain hydrophilic solvent in an amount of 0.1 to 1 wt. %. This hydrophilic solvent must be dermatologically acceptable in the amounts used. Further, solvents with humectant properties are preferably not considered “hydrophilic solvents” in the manner described herein but are ascribed to component (e). The alcohol component (a) is also to be distinguished from the hydrophilic solvent (h). Examples of hydrophilic solvents include alkylene glycols and water-soluble polyalkylene glycols such as propylene glycol, butylene glycol or dipropylene glycol.

Hydrophilic solvents do not constitute an essential component of the antimicrobial cleansing composition but can be added to enhance the solubilisation of individual components. Sometimes, hydrophilic solvents are present, for instance in the commercially available formulation of other components used to formulate the antimicrobial cleansing composition.

As a further optional component (i), the antimicrobial cleansing composition may also comprise pH-regulating substances in an amount of up to 1 wt. %. The pH of the antimicrobial cleansing composition can be 5.0 to 7.0, or 5.2 to 6.5. The pH-regulating substance can be used for adjusting the pH to these values. It can be selected from dermatologically acceptable organic acids such as citric acid or lactic acid.

As a further optional component (j), the antimicrobial cleansing composition may also contain other auxiliary substances which are sometimes used in alcohol-based antimicrobial compositions such as fragrances or dyes. In particular embodiments, the total amount of such auxiliary substances should not exceed 1 wt. %.

In a first embodiment, the antimicrobial cleansing composition is capable of forming foam and includes at least the following ingredients (a) and (b) in the amounts stated below, and water:
(a) 35 to 50 wt. % of an alcohol selected from ethanol and propanol,
(b) 1 to 8 wt. % of a surfactant selected from sulfate salt surfactants and amphoteric surfactants, such as a sulfate salt surfactant including (i) an anionic molecule including a monovalent hydrocarbon group having 8 to 20 carbon atoms, which is linked via an alkyleneoxy unit or polyalkyleneoxy unit with an anionic group and (ii) as cationic counterion an alkyl group having 2 to 4 carbon atoms and including at least one amino group and at least one hydroxyl group substituent.

In one aspect of the first embodiment, the composition further includes:
(b′) optionally up to 5 wt. %, or up to 1 wt. %, e.g. optionally 0.1 to 1.0 wt. % of a secondary surfactant (different from surfactant (b)), said secondary surfactant can be a nonionic co-surfactant,
(c) optionally up to 5 wt. % of an agent selected from antibacterial, antifungal and antiviral agents,
(e) optionally 0.5 to 5 wt. % of a humectant,
(f) optionally 0.5 to 5 wt. % of a film-forming agent,
(g) optionally 0.1 to 5 wt. % of at least one skin care active,
(h) optionally 0.1 to 1 wt. % of a hydrophilic solvent,
(i) optionally up to 1 wt. % of a pH-regulating compound,
(j) optionally up to 1 wt. % of other auxiliary substances, and
(k) water.

Surprisingly, it has been found that this antimicrobial cleansing composition is able to be dispensed as a sufficiently stable foam with the use of non-propellant foam-dispensing devices from non-pressurized containers. It is advantageous to provide the antimicrobial cleansing composition as foam to the user since foams can be spread easily and uniformly over the hands. This contributes to the cleansing efficiency of the composition. Despite the low alcohol content, this embodiment also shows an excellent antimicrobial activity.

Regarding the preferred embodiments and/or the amounts of components (a), (b), (b′), (c), (e), (f), (g), (h), (i) and (j), respectively, reference is made to the above description. Component (c) is not required in the first embodiment but may be added to supplement the spectrum of antibacterial and/or antifungal and/or antiviral activity.

In particular embodiments, the antimicrobial cleansing composition of the first embodiment includes:
(a) 35 to 45 wt. % of ethanol containing not more than 20 wt. % propanol
(b) 2 to 6 wt. % of a surfactant as defined above,
(b′) optionally up to 1 wt. %, e.g. 0.1 to 1 wt. % of a secondary surfactant, said secondary surfactant can be a nonionic co-surfactant
(e) 0.5 to 3 wt. % of a humectant, such as diols or triols such as glycerol,
(g) 0.1 to 5 wt. % of at least one skin care active, such as cell-regenerating agents such as betaine,
(h) optionally 0.1 to 1 wt. % of a hydrophilic solvent,
(i) optionally up to 1 wt. % of a pH-regulating compound, such as cosmetically acceptable organic acids such as citric acid,
(j) optionally up to 1 wt. % of other auxiliary substances selected from perfumes and dyes,
(k) the remainder preferably being water.

In a further embodiment (the second embodiment), an antimicrobial cleansing composition includes:
(a) 30 to 60 wt. % of an alcohol selected from ethanol and propanol,
(b) 0.5 to 10 wt. % of a surfactant as defined above,
(b′) up to 5 wt. %, or up to 1 wt. %, e.g. 0.1 to 1 wt. % of a secondary surfactant (different from surfactant (b)) such as a nonionic co-surfactant
(c) optionally up to 5 wt. % of an agent selected from antibacterial, antifungal and antiviral agents,
(d) 0.1 to 3 wt. % of a thickener,
(e) optionally 0.5 to 5 wt. % of a humectant,
(f) optionally 0.5 to 5 wt. % of a film-forming agent,
(g) optionally 0.1 to 5 wt. % of at least one skin care active,
(h) optionally 0.1 to 1 wt. % of a hydrophilic solvent,
(i) optionally up to 1 wt. % of a pH-regulating compound,
(j) optionally up to 1 wt. % of other auxiliary substances, and
(k) water.

During hand wash, this composition generates foam, albeit to a lesser degree than the first embodiment.

Regarding the preferred embodiments of these components and/or the preferred amounts thereof, reference is made to the above description.

This composition is not suitable to be dispensed as foam in view of the fact that it contains thickener. The thickener is used to adjust the viscosity of this liquid cleansing composition to 100 to 20,000 mPa·s (measured at 23° C. with a Brookfield-LV viscosimeter equipped with a helipath, spindle T-F, 12 rpm).

The present disclosure also relates to the use of the antimicrobial cleansing composition according to the first embodiment for making foam.

A further aspect relates to a dispenser containing the antimicrobial cleansing composition according to the invention. This dispenser can be a foam dispenser if the foamable antimicrobial composition (the first embodiment) is to be dispensed. Conventional foam dispensers can be used for this purpose.

A suitable kind of conventional foam dispenser includes a dispensing assembly to be mounted on or in an opening of a container for holding a liquid to be dispensed in the form of a foam, i.e. the foamable antimicrobial composition (the first embodiment). The dispensing assembly includes a pump assembly which in turn includes a liquid piston pump and an air piston pump concentrically arranged in relation to each other. The cylinders of the liquid pump and the air pump are formed by the inner cylinder and outer cylinder of a double cylinder, respectively. A liquid piston and an air piston are reciprocally movably arranged in the liquid cylinder and the air cylinder. The dispensing assembly further includes a common actuation member for simultaneous actuation of the liquid pump and the air pump by manual depression of the actuation member: the liquid piston and the air piston are operatively connected to the actuation member so that upon actuation of the actuation member the liquid piston and the air piston are reciprocally moved in the liquid cylinder and the air cylinder, respectively.

When the above dispensing assembly is mounted on the container containing a foamable liquid, i.e. the foamable antimicrobial composition according to the first embodiment, and the actuation member is operated, the liquid and air are compressed in the pump chambers of the liquid pump and the air pump, respectively, and both the liquid and the air are pumped into a mixing chamber to be mixed with each other to form a (pre)foam. The mixture of liquid and air is further pumped through a dispensing channel while passing one or more porous elements, such as sieves, to form a homogeneous foam which is dispensed at a dispensing opening at the end of the dispensing channel.

When the common actuation member is released, the upward stroke of the pump pistons increases the size of the pump chambers so that liquid is drawn from the reservoir into the liquid pump chamber and air is sucked into the air pump chamber. A new amount of foam can now be dispensed by operating the actuating member.

Foam-dispensing assemblies of the type described above are, for instance, described in EP 0 613 728 A2, U.S. Pat. No. 6,053,364, US 2009/0236371 A1 and US2010/0320232 A1, the latter three documents being in the name of REXAM AIRSPRAY N.V., and shown in particular in FIG. 1 of US 2009/0236371 A1, for example. However, the foam dispenser does not require the porous matrix containing a dispensate material being soluble or dispersible in the already generated foam which is present in the dispenser of FIG. 1 of US 2009/0236371 A1.

Dispensing assemblies of this type are commercially available from REXAM AIRSPRAY N.V. and are suitable for use with the present disclosure.

### EXAMPLES

The following examples illustrate embodiments of the present invention.

### Example 1

### Antimicrobial Cleansing Composition

A foamable antimicrobial cleansing composition was prepared at room temperature by mixing water and alcohol and dissolving the remaining components in the mixture.
40 wt. % denatured alcohol (36 wt. % ethanol/4 wt. % isopropyl alcohol as denaturation agent)
4.0 wt. % of a solution of 85 wt. % MIPA-C12-14 fatty alcohol polyethyleneglycolether sulfate and 5 wt. % ethoxylated C12-C14 fatty alcohols in 10 wt. % propylene glycol (Marlinat® 242/90 from Sasol Germany GmbH O&S)
1 wt. % glycerin
1 wt. % betaine
≦0.5 wt. % citric acid (to adjust the pH to 5 to 6)
ad 100 wt. % aqua

After mixing a transparent aqueous solution was obtained.

Using a conventional non-propellant foam-dispensing device, this antimicrobial cleansing composition yielded a foam which was sufficiently stable (at least for about 20 seconds) for washing the hands.

This was unexpected since MIPA-laureth sulfate is typically used in water-free formulations (e.g. bath oils) and is known for having good perfume and oil emulsifying characteristics which are not related to the effects achieved by embodiments of the present invention.

This antimicrobial foam cleanser was evaluated in various tests as follows:

In an epicutaneous 24 hour patch test with 22 test persons, the skin tolerability of the foam cleanser was found to be very good. The mean irritation scores were within the range of the negative control (aqua demineralised) at all readings.

The bacterial efficacy of the foam cleanser in the quantitative suspension test according to DIN EN 1040 (status December 2005) on the test organisms Staphylococcus aureus ATCC 6538 and Pseudomonas aeruginosa ATCC 15442 was confirmed (reduction ≧5.0 lg).

Further, the antimicrobial foam cleanser was subjected to a hygienic hand wash test according to DIN EN 1499 based on the following wash procedure:

1. Moisten the hands with warm water at 30° C. for about 10 seconds.

2. Wash thoroughly for 2×30 seconds using 2× four pump strokes (2×1.5 g) of foam cleanser. Massage the soap on both sides of the hands and between the fingers for 60 seconds, in accordance with the standard procedure.

3. Rinse the hands with water at 30° C. for 30 seconds.

4. Dry the hands using a paper towel until the skin feels dry.

The pH of the foam cleanser was 5.37 and the test strain Escherichia (E.) coli K12 (NCTC 10538). Using the reference hand wash, the hands were washed with 1×5 ml Kalisoap within 1 minute, immediately after the determination of the pre-values. The foam cleanser of embodiments of the invention showed a mean log reduction of 3.30 log (n=15) compared to a mean log reduction of 2.86 log (n=15) for the reference product. Therefore, the antimicrobial foam cleanser fulfils its function as a product for hygienic hand washing, in accordance with the requirements of EN 1499.

In accordance with the requirements of EN 1276, it was also found that the antimicrobial foam cleanser showed a bacterial efficacy with respect to the tested strains shown below at a concentration of 75% after a contact time of 5 minutes under dirty conditions and at a test temperature of 20° C.:

| | |
|---|---|
| Staphylococcus (S.) aureus | ATCC 6538 |
| Escherichia (E.) coli | ATCC 10536 |
| Enterococcus (Ec.) hirae | ATCC 10541 |
| Pseudomonas (P.) aeruginosa | ATCC 15442 |

In the evaluation of the fungicidal activity according to EN 1275, the antimicrobial foam cleanser showed a so-called “yeasticidal” activity with reference to the tested strain Candida albicans (ATCC 10231) because it passed the required criteria of >4.0 log reduction after a contact time of 15 min. The log reduction at the same concentration with reference to Aspergillus brasiliensis (ATCC 16404) was however only log 0.44.

The virus-inactivating properties of the antimicrobial foam cleanser against Vaccinia virus and Bovine Viral Diarrhea Virus (BVDV, surrogate of Hepatitis C Virus) were tested with an “undiluted” solution of the foam cleanser resulting from the addition of 20 parts by weight test virus suspension to 80 parts by weight foam cleanser. It was found that an exposure time of 30 seconds was required in order to achieve a four log₁₀ reduction (inactivation ≧99.99%) in a quantitative suspension test according to the guideline of the Deutsche Vereinigung zur Bekämpfung der Viruskrankheiten e.V. (DVV, German Association for the Control of Virus Diseases) and the Robert-Koch-Institute (RKI).

After evaluation with Vaccinia virus and BVDV, the antimicrobial Foam Cleanser can be declared as having “limited virucidal” properties according to a recommendation of an expert committee of RKI (Bundesgesundheitsbl 2004, 47:62-66) and is thus able to inactivate all enveloped viruses.

Therefore, after successful experiments with the two above mentioned enveloped viruses the antimicrobial Foam Cleanser is also effective against the so-called blood-born viruses including HBV, HCV and HIV as well as against members of other virus families such as orthomyxoviridae (incl. all human and animal influenza viruses like H5N1 and H1N1).

### Examples 2 to 6

The antimicrobial cleansing composition of Example 1 was modified by replacing 4.0 wt. % of Marlinat® 242/90 by 2.0 wt. % of one of the following surfactants:
Example 2: Mackadet® from Rhodia UK Ltd., i.e. MIPA-laureth sulphate (33-39 wt. %), laureth-3 (32-38 wt. %) and cocamide MIPA (12-16 wt. %) dissolved in butylene glycol and propylene glycol.
Example 3: Sodium laureth sulphate
Example 4: Sodium lauryl sulfate
Example 5: Cocamidopropyl betaine
Example 6: Sodium cocoamphoacetate

These compositions resulted in less stable but still acceptable foams when compared with example 1. The foam of Example 2 was almost as good as that of example 1, followed by the foams of example 3 and 4, respectively.

### Comparative Examples 1, 2 and 3

The antimicrobial cleansing composition of Example 1 was modified by replacing 4.0 wt. % of Madinat® 242/90 by 2.0 wt. % of one of the following surfactants:
Comparative Example 1: Coco glucoside and disodium lauryl sulfosuccinate
Comparative Example 2: Coco glucoside
Comparative Example 3: Lauramine oxide

The stability of the foams generated by these comparative products was rated poor.

1. An antimicrobial cleansing composition comprising
(a) 20 to less than 60 wt. % of at least one alcohol selected from ethanol and propanol,
(b) 0.5 to 10 wt. % of a surfactant selected from sulfate salt surfactants and amphoteric surfactants, and
water.

2. The antimicrobial cleansing composition according to claim 1, wherein surfactant (b) is a sulfate salt surfactant and comprises (i) an anionic molecule comprising a monovalent hydrocarbon group having 8 to 20 carbon atoms, which is linked via an alkyleneoxy unit or polyalkyleneoxy unit with a sulfate group and (ii) as cationic counter-ion a protonated amine.

3. The antimicrobial cleansing composition according to claim 1, wherein the cationic counter-ion of the surfactant comprises propane substituted by one amino group and one hydroxyl group.

4. The antimicrobial cleansing composition according to claim 1, further comprising one or more components selected from secondary surfactants, antibacterial agents, antiviral agents, antifungal agents, thickening agents, humectant agents, film-forming agents, skin care actives, hydrophilic solvents, pH-regulating substances and other auxiliary agents.

5. The antimicrobial cleansing composition according to claim 1 having a pH of 5 to 7.

6. The antimicrobial cleansing composition according to claim 1 further comprising at least one of
(b′) up to 5 wt. % of a secondary surfactant,
(c) up to 5 wt. % of an agent selected from antibacterial, antifungal and antiviral agents,
(d) 0.1 to 3 wt. % of a thickener,
(e) 0.5 to 5 wt. % of a humectant agent,
(f) 0.5 to 5 wt. % of a film-forming agent,
(g) 0.1 to 5 wt. % of at least one skin care active,
(h) 0.1 to 3 wt. % of a hydrophilic solvent,
(i) up to 1 wt. % of a pH-regulating compound, and
(j) up to 1 wt. % of other auxiliary substances.

7. The antimicrobial cleansing composition according to claim 6, wherein
(d) the thickener is selected from cellulose ethers,
(e) the humectant agent is selected from polyols,
(f) the film-forming agent is selected from ethanol-soluble oils,
(g) the skin care active is selected from cell-regenerating agents,
(i) the pH-regulating compound is selected from organic acids, and
(j) other auxiliary substances are selected from perfumes and dyes.

8. The antimicrobial cleansing composition according to claim 1, comprising
(a) 35 to 50 wt. % of the at least one alcohol selected from ethanol and propanol,
(b) 1 to 8 wt. % of the surfactant,
(b′) optionally up to 5 wt. % of a secondary surfactant,
(c) optionally up to 5 wt. % of an agent selected from antibacterial, antifungal and antiviral agents,
(e) 0.5 to 5 wt. % of a humectant agent,
(f) optionally 0.5 to 5 wt. % of a film-forming agent,
(g) 0.1 to 5 wt. % of at least one skin care active,
(h) optionally 0.1 to 3 wt. % of a hydrophilic solvent,
(i) optionally up to 1 wt. % of a pH-regulating compound,
(j) optionally up to 1 wt. % of other auxiliary substances, and the remainder being water.

9. The antimicrobial cleansing composition according to claim 8, comprising
(a) 35 to 45 wt. % of ethanol containing not more than 20 wt. % propanol,
(b) 2 to 6 wt. % of the surfactant,
(b′) optionally up to 1 wt. % of a secondary surfactant,
(e) 0.5 to 3 wt. % of a humectant,
(g) 0.1 to 5 wt. % of at least one skin care active,
(h) optionally 0.1 to 1 wt. % of a hydrophilic solvent,
(i) optionally up to 1 wt. % of a pH-regulating compound,
(j) optionally up to 1 wt. % of other auxiliary substances selected from perfumes and dyes,
the remainder being water.

10. The antimicrobial cleansing composition according to claim 1, further comprising
(b′) optionally up to 5 wt. % of a secondary surfactant,
(c) optionally up to 5 wt. % of an agent selected from antibacterial, antifungal and antiviral agents,
(d) 0.1 to 3 wt. % of a thickener,
(e) 0.5 to 5 wt. % of a humectant,
(f) optionally 0.5 to 5 wt. % of a film-forming agent,
(g) 0.1 to 5 wt. % of at least one skin care active,
(h) optionally 0.1 to 3 wt. % of a hydrophilic solvent,
(i) optionally up to 1 wt. % of a pH-regulating compound,
(j) optionally up to 1 wt. % of other auxiliary substances,
the remainder being water.

11. The antimicrobial cleansing composition according to claim 10, wherein the thickener is selected from cellulose ether and the composition has a viscosity of 100 to 20000 measured with a Brookfield LV viscosimeter at 23° C., spindle T-F, 12 rpm.

12. A method of using the antimicrobial cleansing composition of claim 8 comprising the step of making a foam.

13. A dispenser comprising the antimicrobial cleansing composition of claims 1.

14. A foam soap dispenser comprising the antimicrobial cleansing composition of claim 8.

15. The foam soap dispenser according to claim 14, comprising a container for accommodating the antimicrobial cleansing composition and a dispensing assembly to be mounted on or in an opening of the container,
wherein the dispensing assembly includes a pump assembly comprising a liquid piston pump and an air piston pump, and a common actuation member for simultaneously actuating the liquid pump and the air pump,
and wherein the pump assembly includes a mixing chamber for mixing the antimicrobial composition with air, and a dispensing channel which includes one or more porous elements for forming a homogeneous foam from the mixture and a dispensing opening for dispensing the foam.

16. The antimicrobial cleansing composition according to claim 2, wherein the protonated amine is a protonated mono-, di- or tri-hydroxyalkylamine.

17. The antimicrobial cleansing composition according to claim 2, wherein the anionic molecule of the sulphate salt surfactant comprises a C10 to C14 alkyl group which is linked via a (O—CH₂—CH₂)ₙ unit with the sulphate group, wherein the average value of n is 1 to 6.

18. The antimicrobial cleansing composition according to claim 6, wherein the composition comprises up to 5 wt. % of a non-ionic co-surfactant as the secondary surfactant.

19. The antimicrobial cleansing composition according to claim 7, wherein the composition comprises 0.5 to 5 wt. % of glycerol as the humectant agent.

20. The antimicrobial cleansing composition according to claim 7, wherein the composition comprises 0.5 to 5 wt. % of silicone as the film-forming agent.

21. The antimicrobial cleansing composition according to claim 7, wherein the composition comprises 0.1 to 5 wt. % of betaine as the skin care active.

22. The antimicrobial cleansing composition according to claim 7, wherein the composition comprises up to 1 wt. % of citric acid as the pH-regulating compound.

23. The antimicrobial cleansing composition according to claim 9, wherein the composition comprises up to 1 wt. % of a non-ionic co-surfactant as the secondary surfactant.

24. The antimicrobial cleansing composition according to claim 9, wherein the humectant agent is glycerol.

25. The antimicrobial cleansing composition according to claim 9, wherein the skin care active is betaine.

26. The antimicrobial cleansing composition according to claim 9, wherein the pH-regulating compound is an organic acid.

27. The antimicrobial cleansing composition according to claim 10, wherein the composition comprises up to 5 wt. % of a non-ionic co-surfactant as the secondary surfactant.
